(19) United States
(12) Patent Application Publication
(10) Pub. No.: US 2025/0384705 A1
(43) Pub. Date: Dec. 18, 2025

(54) SYSTEM AND METHOD FOR BIOMARKER DETECTION

(71) Applicants: Hoffmann-La Roche Inc., Little Falls, NJ (US); Roche Sequencing Solutions, Inc., Pleasanton, CA (US)

(72) Inventors: Samaneh ABBASI-SURESHJANI, Basel (CH); Christian DOERIG, Basel (CH); Anil YÜCE, Basel (CH); Markus Daniel HERRMANN, Basel (CH); Konstanty Korski, Basel (CH); Jacob GILDENBLAT, Basel (CH); Agata MOSINSKA, Basel (CH); Ping-Chang LIN, Pleasanton, CA (US); Nazim SHAIKH, Pleasanton, CA (US); Qiangqiang GU, Pleasanton, CA (US)

(21) Appl. No.: 19/313,152

(22) Filed: Aug. 28, 2025

Related U.S. Application Data
(63) Continuation of application No. PCT/US2024/018390, filed on Mar. 4, 2024.
(60) Provisional application No. 63/488,253, filed on Mar. 3, 2023.
(60) Provisional application No. 63/506,866, filed on Jun. 8, 2023.
(60) Provisional application No. 63/507,704, filed on Jun. 12, 2023.
(60) Provisional application No. 63/515,655, filed on Jul. 26, 2023.

(51) Int. Cl. G06V 20/69; G06N 3/045; G06T 7/00; G06V 10/82; G16H 30/40

(52) U.S. Cl. CPC G06V 20/698; G06N 3/045; G06T 7/0012; G06V 10/82; G06V 20/695; G16H 30/40; G06T 2207/30024

(57) ABSTRACT

A method of detecting a biomarker by a detection system based on machine learning includes identifying, by the detection system, a plurality of tiles corresponding to whole-slide image data of a tissue sample; generating, by the detection system, tile-level embeddings data based on the plurality of tiles; generating, by the detection system, cell-level embeddings data based on the plurality of tiles; and generating, by the detection system, a slide-level prediction based on the tile-level embeddings data and the cell-level embeddings data, the slide-level prediction indicating presence or absence of the biomarker in the tissue sample.

### CROSS-REFERENCE TO RELATED APPLICATION(S)

This application is a continuation of International Application No. PCT/US2024/018390, (SYSTEM AND METHOD FOR BIOMARKER DETECTION), filed Mar. 4, 2024, which claims priority to, and the benefit of, U.S. Provisional Application Nos. 63/488,253 (“EXPLAINABLE PLUG AND PLAY FOR FEATURE REPRESENTATION IN HISTOPATHOLOGY”), filed on Mar. 3, 2023, U.S. Provisional Application No. 63/506,866 (“CELL OF ORIGIN PREDICTION FOR DIFFUSE LARGE B CELL LYMPHOMAS”), filed on Jun. 8, 2023, U.S. Provisional Application No. 63/507,704 (“CELL OF ORIGIN PREDICTION FOR DIFFUSE LARGE B CELL LYMPHOMAS”), filed on Jun. 12, 2023, and U.S. Provisional Application No. 63/515,655 (“DEEP LEARNING BASED WHOLE SLIDE IMAGE ANALYSIS FOR IDENTIFICATION OF MYC-DRIVEN HIGH-GRADE B-CELL LYMPHOMA”), filed on Jul. 26, 2023, the entire contents of which are incorporated herein by reference.

### FIELD

Aspects of some embodiments of the present disclosure relate to a system and method for biomarker detection.

### BACKGROUND

Cancers in their various forms have become one of the leading causes of death worldwide. Early diagnosis plays an important role in achieving the best treatment outcomes for people with cancer. Identification of cancer biomarkers permits more granular classification of tumors, leading to better diagnosis and prognosis, and enabling more informed treatment decisions. For many cancers, clinically viable and reliable biomarkers have still not been identified and biomarker identification techniques have limitations that can restrict their clinical use. On the other hand, histological analysis of hematoxylin and eosin (H&E) stained pathology slides is widely used in cancer diagnosis and prognosis. However, visual examination of H&E-stained slides is insufficient for classification of some tumors because morphological differences that may discriminate between subtypes are beyond the limits of human detection.

The above information disclosed in this Background section is only for enhancement of understanding of the background and therefore the information discussed in this Background section does not necessarily constitute prior art.

### SUMMARY

Aspects of some embodiments of the present disclosure are directed to a biomarker detection system that extracts both tile-level and cell-level embeddings data from a WSI and combines the embeddings to simultaneously capture histology and cytology features and improve model performance and explainability. As a result, the detection system is capable of making more accurate predictions with regards to presence of particular biomarkers in a sample represented by the WSI.

According to some embodiments of the present disclosure, there is provided a method of detecting a biomarker by a detection system based on machine learning, the method including: identifying, by the detection system, a plurality of tiles corresponding to whole-slide image data of a tissue sample; generating, by the detection system, tile-level embeddings data based on the plurality of tiles; generating, by the detection system, cell-level embeddings data based on the plurality of tiles; and generating, by the detection system, a slide-level prediction based on the tile-level embeddings data and the cell-level embeddings data, the slide-level prediction indicating presence or absence of the biomarker in the tissue sample.

In some embodiments, the identifying the plurality of tiles includes receiving, by the detection system, the whole-slide image data corresponding to the tissue sample; and extracting, by the detection system, the plurality of tiles from the whole-slide image data.

In some embodiments, the whole-slide image data includes at least one a digitized image of the tissue sample of a patient that is stained with hematoxylin and eosin (H&E) dyes or a region-of-interest (ROI) map.

In some embodiments, the method further includes performing stain normalizing, by the detection system, based on the plurality of tiles to generate a plurality of normalized tiles, wherein the generating the tile-level embeddings data includes generating, by the detection system, the tile-level embeddings data from the plurality of normalized tiles.

In some embodiments, the performing stain normalizing includes generating, by a first model of the detection system, the plurality of normalized tiles based on the plurality of tiles.

In some embodiments, the first model includes a fully convolutional neural network.

In some embodiments, the generating the tile-level embeddings data includes generating, by a second model of the detection system, a plurality of tile-level feature vectors based on the plurality of tiles, and wherein a number of the tile-level feature vectors corresponds to a number of the tiles.

In some embodiments, the second model includes at least one of a residual network (ResNet) or a transformer network, and wherein the number of the tile-level feature vectors is a same as the number of the tiles.

In some embodiments, the generating the cell-level embeddings data includes extracting, by the detection system, a plurality of cell patches based on the plurality of tiles; and generating, by the detection system, the cell-level embeddings data based on the plurality of cell patches.

In some embodiments, the extracting the plurality of cell patches includes: detecting, by a segmentation model of the detection system, a plurality of cells in each one of the plurality of tiles; and generating, by the detection system, the plurality of cell patches based on the plurality of tiles and the plurality of cells in each one of the plurality of tiles, a cell patch of the plurality of cell patches includes a portion of one of the plurality of tiles containing a single cell of the plurality of cells.

In some embodiments, the extracting the plurality of cell patches includes extracting, by the detection system, the plurality of cell patches from a plurality of normalized tiles corresponding to the plurality of tiles.

In some embodiments, the generating the cell-level embeddings data includes generating, by a third model of the detection system, a plurality of cell-level feature vectors based on the plurality of cell patches, and wherein a number of the cell-level feature vectors corresponds to a number of the plurality of tiles and a number of the cell patches.

In some embodiments, the third model includes at least one of a residual network (ResNet) or a transformer network, and wherein the number of the cell-level feature vectors is a number of the plurality of tiles multiplied by a number of the cell patches.

In some embodiments, the generating the cell-level embeddings data further includes combining, by the detection system, the plurality of cell-level feature vectors to generate the cell-level embeddings data, the cell-level embeddings data including a plurality of embedding vectors, and wherein a number of the embedding vectors corresponds to a number of the plurality of tiles.

In some embodiments, an embedding vector of the plurality of embedding vectors includes an average of a number of the plurality of cell-level feature vectors and a standard deviation of the number of the plurality of cell-level feature vectors.

In some embodiments, the method further includes aggregating, by the detection system, the tile-level embeddings data and the cell-level embeddings data to generate aggregate embeddings data, wherein generating the slide-level prediction is by a fourth model of the detection system and is based on the aggregate embeddings data.

In some embodiments, the aggregating the tile-level embeddings data and the cell-level embeddings data includes concatenating, by the detection system, the tile-level embeddings data and the cell-level embeddings data to generate the aggregate embeddings data, a vector length of the aggregate embeddings data is equal to a sum of vector lengths of the tile-level embeddings data and the cell-level embeddings data.

In some embodiments, the fourth model includes at least one of a multiple-instance learning (MIL) network, an attention-based MIL (AMIL) network, or a transformer.

In some embodiments, the slide-level prediction includes an MYC-driven high-grade B-cell lymphoma (HGBL) signature.

In some embodiments, the method further includes transmitting the slide-level prediction to a display device for display to a user.

According to some embodiments of the present disclosure, there is provided a detection system for detecting a biomarker, the detection system including: a processor; and a memory storing instructions that, when executed on the processor, cause the processor to perform: identifying a plurality of tiles corresponding to whole-slide image data of a tissue sample; generating tile-level embeddings data based on the plurality of tiles; generating cell-level embeddings data based on the plurality of tiles; and generating a slide-level prediction based on the tile-level embeddings data and the cell-level embeddings data, the slide-level prediction indicating presence or absence of the biomarker in the tissue sample.

In some embodiments, the identifying the plurality of tiles includes: receiving the whole-slide image data corresponding to the tissue sample; and extracting the plurality of tiles from the whole-slide image data, and wherein the whole-slide image data includes at least one a digitized image of the tissue sample of a patient that is stained with hematoxylin and eosin (H&E) dyes or a region-of-interest (ROI) map.

In some embodiments, the generating the tile-level embeddings data includes generating a plurality of tile-level feature vectors based on the plurality of tiles, and wherein a number of the tile-level feature vectors corresponds to a number of the tiles.

In some embodiments, the detection system further includes performing stain normalizing based on the plurality of tiles to generate a plurality of normalized tiles, wherein the generating the tile-level embeddings data includes: generating the tile-level embeddings data from the plurality of normalized tiles.

In some embodiments, the generating the cell-level embeddings data includes extracting a plurality of cell patches based on the plurality of tiles; and generating the cell-level embeddings data based on the plurality of cell patches.

In some embodiments, the extracting the plurality of cell patches includes: detecting a plurality of cells in each one of the plurality of tiles; and generating the plurality of cell patches based on the plurality of tiles and the plurality of cells in each one of the plurality of tiles, a cell patch of the plurality of cell patches includes a portion of one of the plurality of tiles containing a single cell of the plurality of cells.

In some embodiments, the generating the cell-level embeddings data includes generating a plurality of cell-level feature vectors based on the plurality of cell patches, and wherein a number of the cell-level feature vectors corresponds to a number of the plurality of tiles and a number of the cell patches.

In some embodiments, the generating the cell-level embeddings data further includes: combining the plurality of cell-level feature vectors to generate the cell-level embeddings data, the cell-level embeddings data including a plurality of embedding vectors, wherein a number of the embedding vectors corresponds to a number of the plurality of tiles, and wherein an embedding vector of the plurality of embedding vectors includes an average of a number of the plurality of cell-level feature vectors and a standard deviation of the number of the plurality of cell-level feature vectors.

In some embodiments, the detection system further includes aggregating the tile-level embeddings data and the cell-level embeddings data to generate aggregate embeddings data, wherein generating the slide-level prediction is by a fourth model of the detection system and is based on the aggregate embeddings data.

In some embodiments, the aggregating the tile-level embeddings data and the cell-level embeddings data includes: concatenating the tile-level embeddings data and the cell-level embeddings data to generate the aggregate embeddings data, a vector length of the aggregate embeddings data is equal to a sum of vector lengths of the tile-level embeddings data and the cell-level embeddings data.

### BRIEF DESCRIPTION OF THE DRAWINGS

Non-limiting and non-exhaustive embodiments according to the present disclosure are described with reference to the following figures, wherein like reference numerals refer to like parts throughout the various views unless otherwise specified.

FIG. 1 is a block diagram illustrating a biomarker detection system, according to some embodiments of the present disclosure.

FIG. 2 is a block diagram illustrating the whole-slide image (WSI) preprocessor and its operation, according to some embodiments of the present disclosure.

FIG. 3A is a block diagram illustrating the internal structure of the tile-level analyzer and the cell-level analyzer of the biomarker detection system, according to some embodiments of the present disclosure.

FIG. 3B is a block diagram illustrating the cell patch extractor of the cell-level analyzer, according to some embodiments of the present disclosure.

FIG. 4 is a flow diagram illustrating a process of detecting a biomarker by the biomarker detection system, according to some embodiments of the present disclosure.

### DETAILED DESCRIPTION

Hereinafter, aspects of some example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

In general, testing for cancer biomarkers can improve the accuracy of tumor classification, which leads to better diagnosis, prognosis and treatment decisions. However, biomarker testing can be time-consuming and unavailable in some settings. Visual examination of hematoxylin and eosin (H&E)-stained pathology slides is widely used in cancer diagnosis and prognosis. However, this may be insufficient to classify some tumors because morphological differences between molecularly defined subtypes may be beyond the limit of human detection.

The introduction of digital pathology (DP) has enabled the application of machine learning (ML) approaches to extract otherwise inaccessible diagnostic and prognostic information from H&E-stained whole slide images (WSIs). Current ML approaches use embeddings derived from slide-level aggregations of data, extracted across multiple tiles of the WSI that each contain many cells, and these often fail to capture useful information from individual cells in each tile.

Aspects of some embodiments of the present disclosure are directed to a biomarker detection system that extracts both tile-level and cell-level embeddings from a WSI and combines the embeddings to simultaneously capture histology and cytology features and improve model performance and explainability. As a result, the detection system is capable of making more accurate predictions with regards to presence of particular biomarkers in a sample represented by the WSI.

As an example, the detection system may be utilized to identify MYC-driven high-grade B-cell lymphoma (HGBL) based on morphology from H&E-stained WSIs. HGBL is an aggressive lymphoma that often harbors MYC rearrangements (MYC-R) and molecular signatures attributed to aberrant MYC activation. Identifying and classifying HGBL is challenging, and current classification systems recognize diffuse large B-cell lymphoma (DLBCL) or HGBL with MYC and BCL2 rearrangements (MYC-R/BCL2-R; double-hit; defined molecularly) and HGBL—not otherwise specified (defined morphologically), in which MYC-R occur in up to 45% of cases and the double-hit signature (DHITsig) occurs in 54% of cases. Existing methods for molecular classification, such as fluorescence in situ hybridization (FISH), are expensive, time-consuming and not widely available, and morphological classification is subjective and associated with high inter-reader variability.

In such examples, the detection system may be applied to a WSI to extract cytological features from single cells and histomorphological features from larger tissue regions, to quantify high-grade morphology characterized by monomorphic sheets of dense cells with round, intermediately sized nuclei and finely dispersed chromatin, and thus make an accurate prediction regarding the presence of molecular alterations associated with HGBL, such as the MYC-R biomarker. In some examples, the detection system may be used to predict MYC gene rearrangement in Burkitt lymphoma to avoid FISH testing as well or to predict gene expression signatures such as the double-hit signature (DHITsig) or molecular high-grade (MGH) signature in DLBCL/HGBL to avoid expression profiling. In further examples, the detection system utilizes HPS as a biomarker that characterizes/identifies a specific subpopulation of DLBCL/HGBL patients with a shared biology/pathophysiology and enables other applications, such as patient selection or stratification in clinical trials.

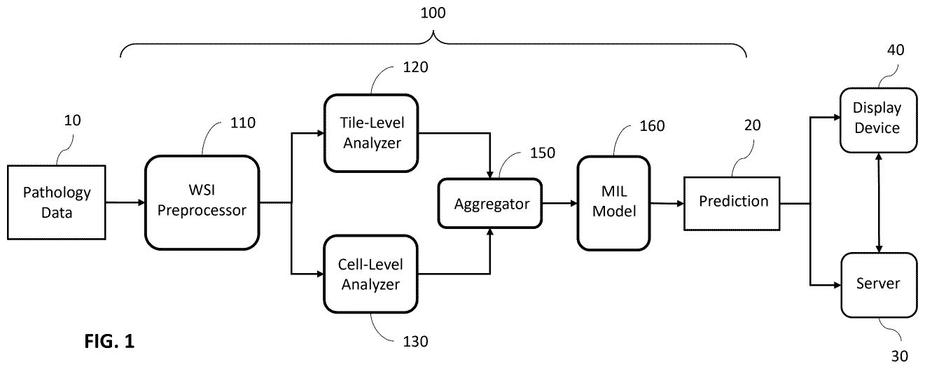

FIG. 1 is a block diagram illustrating the biomarker detection system 100, according to some embodiments of the present disclosure.

According to some embodiments, the biomarker detection system (also referred to as a detection system) 100 is configured to analyze both the tile-level and cell-level features of a given whole slide image (WSI) data 10 and to generate a corresponding prediction 20 regarding the presence or absence of a particular biomarker (such as the MYC-R biomarker). In some examples, the detection system 100 utilizes machine learning-based models to identify MYC-driven HGBL based on morphology from H&E-stained WSIs; however embodiments of the present disclosure are not limited thereto, and the detection system 100 may be utilized to detect or predict the presence of any suitable biomarker, such as a mutation of an individual gene (e.g., loss of function single nucleotide variation in the TP53 gene), a gene mutation signature (e.g., MCD signature based on co-occurrence of MYD88 and CD79B mutations), the expression level of an individual gene or protein (e.g., MYC), a gene expression profile or signature (e.g., cell-of-origin signature), the infiltration of immune cells in the microenvironment (e.g., lymphocytes), and the like.

The WSI data 102 that is supplied to the biomarker detection system 100 may include one or more digitized images of a tissue sample (e.g., a tumorous tissue sample) of the patient that is stained with hematoxylin and eosin dyes. H&E dyes stain cell nuclei, extracellular matrix and cytoplasm, and other cell structures, with different colors thus allowing a pathologist and the detection system 100 to differentiate between different cellular structure. Also, the overall patterns of coloration from the stain show the general layout and distribution of cells and provide a view of a tissue sample's structure. In some examples, the whole-slide image data 102 may include one or more image tiles that are extracted from (e.g., randomly selected and extracted from) a viable tumor region of a stained tissue sample.

The prediction 20 that is output by the biomarker detection system 100 may be a binary output (e.g., ‘0’ or ‘1’, or ‘+’ or ‘−’) indicating the presence or absence of a biomarker for which detection system 100 is trained. In some examples, prediction 20 may be a confidence level or probability that the biomarker is present in the tissue sample associated with the WSI data. However, these are merely examples, and embodiments of the present disclosure are not limited thereto.

According to some embodiments, the biomarker detection system 100 includes a tile-level analyzer 120, a cell-level analyzer 130, and aggregator 150, and a biomarker predictor 160.

In some embodiments, the tile-level analyzer 120 is configured to receive a plurality of tiles corresponding to WSI data 10 of a tissue sample, to analyze the tiles at a tile level, and to generate (e.g., extract) tile-level embeddings data based on the plurality of tiles. The cell-level analyzer 130 also receives the plurality of tiles and is configured to analyze the tiles at a cellular level, and to generate (e.g., extract) cell-level embeddings data based on the plurality of tiles. The aggregator 150 is configured to aggregate (e.g., combine) the tile-level embeddings data and the cell-level embeddings data to generate aggregate embeddings data. The biomarker predictor 160, in turn, generates the slide-level prediction 20 based on the aggregate embeddings data.

Once the biomarker detection system 100 generates a prediction 20, the prediction may be transmitted to a server (e.g., a remote server or a cloud server) 30 for further processing and/or to a display device 40 for display to a user.

Analyzing the WSI data 10 at both a tile-level and a cellular-level can greatly improve the accuracy of the slide-level prediction 20. This is, at least in part, due to the fact that tiles extracted from WSI data 10 may contain different types of cells, as well as non-cellular tissue such as stroma and blood vessels and non-biological features (e.g., glass). When using tile-level embeddings data for prediction, cell density and the proportion of non-cellular tissue per tile can be the dominant predictive factor. Cell-level embeddings may be able to extract useful information, based on the morphological appearance of individual cells, which may be valuable for downstream classification tasks but would otherwise be masked by more dominant features within tile-level embeddings.

In some embodiments, the biomarker detection system 100 also includes a WSI processor 110 that is configured to preprocess the WSI data 10 to ensure uniformity in the tiles that are supplied to the tile-level and cell-level analyzers 120 and 130. Given that different labs that generate whole slide images based on tissue samples may use different stainers and/or settings, the resulting WSIs produced by such labs may have different stains (e.g., different colorations). Therefore, in some embodiments, the WSI processor 110 performs stain normalization, that is, standardizes the stains across all tiles, and generates a plurality of normalized tiles that are then passed onto the tile-level and cell-level analyzers 120 and 130 for further analysis and processing. The WSI processor 110 may also perform the function of extracting tiles from an original WSI.

However, embodiments of the present disclosure are not limited thereto. For example, one or more functions of the WSI processor 110 may be omitted from this component and integrated into other component blocks, or omitted from the biomarker detection system 100 altogether. In some examples, stain normalization may be omitted from the WSI processor 110 and the function may be integrated into the input stage of one more of the tile-level and cell-level analyzers 120 and 130. Further, the stain normalization function may be omitted from the biomarker detection system 100 and each of the tile-level and cell-level analyzers 120 and 130 may operate on the raw tiles with potentially different staining profiles.

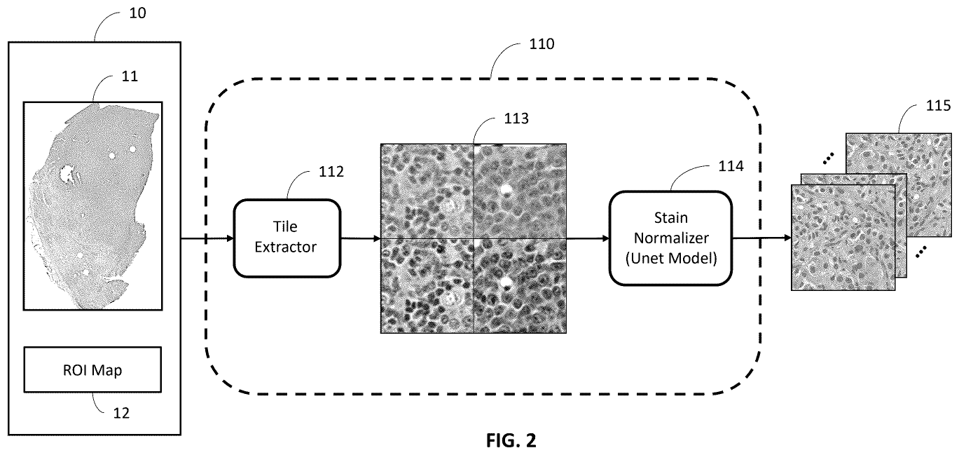

FIG. 2 is a block diagram illustrating the WSI preprocessor 110 and its operation, according to some embodiments of the present disclosure.

In some examples, the WSI data 10 includes a WSI 11 and a region-of-interest (ROI) map 12 that identifies the regions of the WSI 11 that are relevant to the analysis of the biomarker detection system 100. The ROI map 12 may be generated by applying a series of filters to the WSI 11.

In some examples, the filters may include at least one of a background filter, an out-of-focus filter, a crush filter, a pen mark filter, a hemorrhage filter, a necrosis filter, a fat tissue filter, or non-lymphoid filter. The background filter may remove portions of the WSI 11 that do not contain any tissue by detecting portions that contain tissue and discarding everything else. The out-of-focus filter may remove portions of the WSI 11, which contain tissue that are not in focus, i.e., blurry either due to suboptimal image acquisition or slide preparation. The crush filter may remove portions of the WSI 11 that contain tissue with crush artifacts, i.e., clusters of cells that were deformed or damaged due to suboptimal tissue handling. The pen mark filter may remove portions of the WSI 11 that contain pen marks made on the physical glass slide as may be common in anatomical pathology labs. The hemorrhage filter may remove portions of the WSI 11 that contain tissue with signs of bleeding, i.e., excessive extravascular accumulation of red blood cells that obscures tumor tissue. The necrosis filter may remove portions of the WSI 11 that contain necrotic tissue. This can demonstrate a range of features from eosinophilic tissue debris without intact tumor cells to cells with nuclear changes including pyknosis, karyorrhexis, karyolysis and cytoplasmic vacuolization and/or eosinophilia. The fat tissue filter may remove portions of the WSI 11 that contain fat tissue including adipocytes and associated connective tissue. The non-lymphoid tissue filter may remove portions of the WSI 11 that contain lymphoid tissue. The filter may identify lymphoid tissues, which may be encountered at anatomic sites at which lymphomas can occur, e.g., lymphoma tissue, lymph node parenchyma, lymphoid-rich stroma, lymphoid aggregates in nodal and extranodal anatomic sites. Positively classified WSI portions containing lymphoid tissue may be kept and negatively classified areas not containing lymphoid tissue may be removed.

Each of said filters may represents a function parametrized by a convolutional neural network (CNN) that takes a WSI or a portions thereof as input and returns a single Boolean value as output. The CNN model underlying each filter may be trained to identify a specific histologic concept in WSIs and to classify its portions depending on whether the concept is present or absent. An output of zero may mean that the component did not identify the concept in a given WSI portion, and an output of one may means that the filter did identify the concept in the WSI portion.

The application of the above noted filters produces an analysis region of interest as output. This region that may be continuous or may be spread out in multiple parts, i.e., not continuously connected. In some examples, the region identified by the ROI map 12 includes areas enriched with lymphoid elements (e.g., lymphoma tissue, lymph node parenchyma, lymphoid-rich stroma, lymphoid aggregates) that may be encountered in nodal and extranodal anatomic sites. The ROI may be free of artifacts and non-lymphoid tissue (e.g., be free of background regions, out-of-focus regions, crush tissue, pen mark regions, hemorrhage tissue, necrotic tissue, and fat tissue). In some examples, the ROI map 12 may be further examined and modified by a human user (e.g., pathologist) as desired.

In some embodiments, the WSI preprocessor 110 includes a tile extractor 112 and a stain normalizer 114.

The tile extractor 112 may apply the ROI map 12 to (e.g., overlay the ROI map 12 onto) the WSI 11 to identify regions of interest in the WSI 11 and to then extract a plurality of non-overlapping tiles 113 of equal size from the regions of interest in the WSI 11. In some examples, the tile extractor 112 may also extract tiles from the WSI 11 and discard those tiles that do not fall within the ROI (e.g., tiles that have greater than 10% overlap with non-ROI regions). In some examples, the tile extractor 112 may extract a number of (e.g., more than 30,000) non-overlapping tiles of 256×256 pixels from the WSI 11, which may have been digitized at 40× magnification.

To accommodate for the different stains that the tiles 113 may exhibit (as, e.g., represented by tiles 113a, 113b, 113c, and 133d), the stain normalizer 114 standardizes the stains across the plurality of tiles to generate a plurality of normalized tiles 115 that that have a uniform stain irrespective of the stain used in the WSI 11.

In some embodiments, the stain normalizer 114 includes a first model, which may utilize a U-Net architecture having a neural network (e.g., a convolutional neural network) that expresses an input image in short form as a vector and then upscales the image in the desired (e.g., standardized) stain. However, embodiments of the present disclosure are not limited thereto, and the first model of the stain normalizer 114 may use any suitable architecture.

The stain normalizer 114 provides the normalized tiles 115 to the tile-level and cell-level analyzers 120 and 130 for tile-level and cell-level embeddings extraction, respectively.

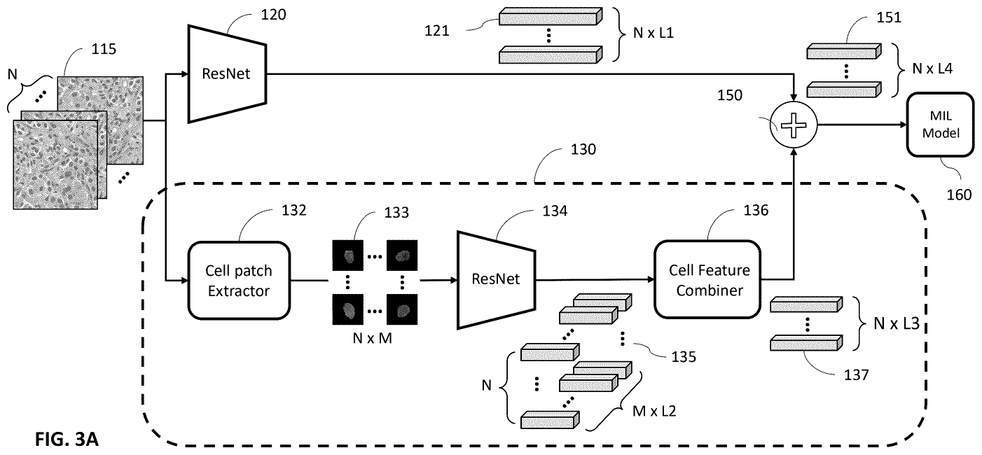

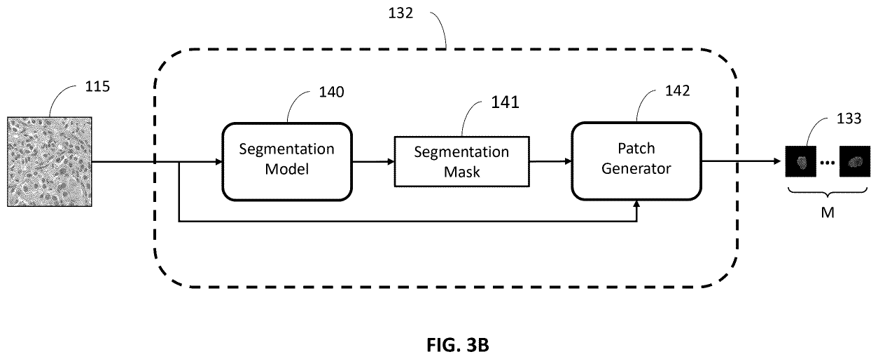

FIG. 3A is a block diagram illustrating the internal structure of the tile-level analyzer 120 and the cell-level analyzer 130 in further detail, according to some embodiments of the present disclosure. FIG. 3B is a block diagram illustrating the cell patch extractor 132 of the cell-level analyzer 130, according to some embodiments of the present disclosure.

Referring to FIG. 3A, in some embodiments, the tile level analyzer 120 includes a second model that receives the plurality of tiles (e.g., non-overlapping normalized tiles) 115 and generates a plurality of tile-level feature vectors 121, as the tile-level embeddings data, based on the received input tiles 115. Each tile-level feature vector 121 may represent measurements of the tissue architectural patterns that are relevant to histopathological diagnosis or biomarker evaluation. Here, the number of tile-level feature vectors 121 (N, an integer greater than 1) corresponds to (e.g., is the same as) the number of the tiles 115 (N). Further, each tile-level feature vector may have a length of L1, which may be 1024, in some examples.

In some embodiments, the second model a convolutional neural network (CNN) architecture, such as a residual network (ResNet) architecture (e.g., ResNet50) or a modified ResNet architecture (e.g., a modified ResNet50). In some examples, the modified ResNet architecture may exclude the last block, i.e., the average pooling, flattening, and fully connected (FC) layers of the ResNet architecture, to improve alignment with cellular content and to increase model interpretability. However, embodiments of the present disclosure are not limited to CNNs, and any suitable neural network, such as a transformer, may be used as the second model.

To enable the use of large unlabeled clinical imaging datasets, the second model may be trained with public and/or proprietary data sets. For example, the modified ResNet50 model may be trained with the bootstrap your own latent (BYOL) method using a number of publicly available datasets from The Cancer Genome Atlas (TCGA): TGCA Breast Invasive Carcinoma (TGCA-BRCA), TGCA Lung Adenocarcinoma (TGCA-LUAD), TGCA Thyroid Cancer (TGCA-THCA), and TGCA Diffuse Large B-cell Lymphoma (TGCA-DLBCL) datasets. In some examples, the second model may also be trained using one or more datasets from private vendors that include different tissues, tumor types, and diseases including breast, lung, thyroid, lymph node and tonsil tissue and cancers including follicular lymphoma and DLBCL.

Referring still to FIG. 3A, in some embodiments, the cell-level analyzer 120 is configured to extracting a plurality of cell patches based on the plurality of tiles 115, and to generate the cell-level embeddings data based on the plurality of cell patches. According to some embodiments, the cell-level analyzer 120 includes a cell patch extractor 132, a cell feature generator 134, and a cell feature combiner 136.

In some embodiments, the cell patch extractor 132 is configured to receive the plurality of tiles (e.g., N normalized tiles) 115 and to extract the plurality of cell patches (e.g., M patches) 133 from each tile 115. The cell feature generator 134 is configured to generate a plurality of cell-level feature vectors 135 based on the extracted cell patches 133. The cell feature combiner 136, in turn, combines the cell-level feature vectors 135 to generate the cell-level embeddings data.

Referring to FIG. 3B, according to some embodiments, the cell patch extractor 132 includes a segmentation model 140 and a patch generator 142.

In some embodiments, the segmentation model 140 is configured to detect cells (e.g., the cell nuclei) within a tile 115 and to generate a segmentation mask 141 that defines the contours of each cell and effectively separates each cell (e.g., nuclei) from the background. The segmentation model 140 may be deep learning based neural network trained for object detection, such as a StarDist network that is trained to distinguish between cells (e.g., nuclei) and background.

The patch generator 142 applies the segmentation mask 141 to the corresponding tile 115 and generates (extracts) a plurality of patches (e.g., non-overlapping and equal-sized patches) 133 from the corresponding tile 115, such that each patch includes a single cell near or at its center. In some examples, each patch 133 may be a 32×32 pixel image crop centered around a segmented nucleus. The patch generator 142 may also remove the background that surrounds a cell in each patch 133, i.e., set their pixel values to black (RGB 0,0,0). The patch generator 142 may produce M (an integer greater than 1) patches 133 based on each tile that include some or all of the cells detected by the segmentation model 140.

While FIG. 3B illustrates the cell patch extractor 132 according to some examples, embodiments of the present disclosure are not limited thereto. For example, the cell patch extractor 132 may merely apply a fixed grid to the tile 115 and subdivide it into a plurality of equal-sized patches, some of which may not include a cell or may include only a partial cell.

Referring again to FIG. 3, the cell feature generator 134 includes a third model that is the same as or substantially similar to the second model of the tile level analyzer 120 and may extract cell-level features in a similar manner to the extraction of the tile-level features outlined above. For example, the second and third models may be trained by the same data, or the second model of the tile level analyzer 120 may be trained on tile data, while the third model of the cell feature generator 134 is trained on cell data.

In some examples, the third model receives N×M cell patches 133 and generate a corresponding number of cell-level feature vectors 135 (N×M vectors) of length L2 (e.g., 256). That is, the cell feature generator 13 may produce one feature vector 135 for every cell patch 133.

In some examples, the cell-patch images 133 may be shrunk by a factor of 32 by the backbone ResNet50 model so that the 32×32 pixel images have a 1:1 spatial resolution in the output from the tensor. To ensure the cell-level embeddings contain features relevant to the cells, prior to the mean pooling in ResNet50, the spatial image resolution may be increased to 16×16 pixels in the output from the CNN of ResNet50 by enlarging the 32×32 pixel cell-patch images to 128×128 pixels and skipping the last 4 blocks in the Resnet50 network.

Because of heterogeneity in the size of cells detected, each 32×32 pixel cell-patch image may contain different proportions of cellular and non-cellular features. Higher proportions of non-cellular features in an image may cause the resultant embeddings to be dominated by non-cellular tissue features or other background features. Therefore, to limit the information used to create the cell-level embeddings to only cellular features, the cell patch extractor 132 may remove portions of the cell-patch images 133 that are outside of the segmented nuclei by setting their pixel values to black (RGB 0,0,0). Finally, in some embodiments, to prevent the size of individual nuclei or amount of background in each cell-patch image from dominating over the cell-level features, the Global Average Pooling layer of the cell feature generator 134 (e.g., the modified ResNet50) only averages the features inside the boundary of the segmented nuclei, rather than averaging across the whole output tensor from the CNN layers. This allows the cell feature generator 134 to focus on the shape and colorations of the nucleus itself, and not on the surrounding background (the information that could be extracted from the surrounding background may already be captured by the tile-level analysis).

According to some embodiments, the cell feature combiner 136 is configured to combine the plurality of cell-level feature vectors 135 to generate the cell-level embeddings data, which includes a plurality of embedding vectors 137 of length L3 (e.g., 512). The number of the embedding vectors 137 may correspond to (e.g., be equal to) the number of tiles 115 (N).

The cell feature combiner 136 may apply one or more statistical measures to the cell-level feature vectors 135 associated with the same tile 115 to characterize the population. In some embodiments, the cell feature combiner 136 may calculate the mean/median/average and standard deviation across cell-level feature vectors 135 of one tile 115 to generate one mean/median/average vector (of, e.g., length 256) and one standard deviation vector (of, e.g., length 256) per tile 155. The cell feature combiner 136 may concatenate the two resulting vectors to form the embedding vectors 137. Thus, each embedding vector 137 may include an average, median, or mean vector of a number (e.g., M) of the cell-level feature vectors 135 that correspond to the same tile 115 and a standard deviation of the same set of cell-level feature vectors 135.

However, embodiments of the present disclosure are not limited thereto. For example, instead of performing the above-noted statistical calculations, the cell feature combiner 136 may include an attention block that operates at the tile level and determines how important each tile 115 is, and assigns a corresponding weight to the cell-level feature vectors 135 of the same tile 115 and combines them by adding the weighted cell-level feature vectors 135 across tiles.

In some embodiments, the aggregator 150 concatenates the tile-level embeddings data and the cell-level embeddings data to generate the aggregate embeddings data 151. Here, a vector length L4 of the aggregate embeddings data 151 may be equal to a sum of vector lengths of the tile-level embeddings data and the cell-level embeddings data. In some examples, the mean and standard deviation of the vectors of the cell-level embeddings for each tile that are concatenated to each corresponding tile-level embedding may result in a combined embedding representation with a total size of 1536 pixels (1024+256+256).

According to some embodiments, the biomarker predictor 160 includes a fourth model with a multiple instance learning framework, which generates the WSI-level prediction 20 based on the aggregate embeddings data 151. In some examples, the fourth model may include a multiple instance learning (MIL) network based on a softmax attention or a transformer attention mechanism, a weakly supervised classifier with maximum, minimum, or average pooling, a graph convolutional network, and/or the like.

In some examples, the prediction 20 may be a continuous probabilistic score that represents the probability for the presence of a biomarker (e.g., an MYC gene rearrangement) in the tumor cells contained in the WSI data 10. The predicted score may be further thresholded at a predetermined threshold to classify the WSI into one of two possible results: biomarker negative when the score is below the threshold or biomarker positive when the score is equal to or above the threshold.

In some examples, the biomarker detection system 100 may be used as an adjunctive aid to rule out MYC rearrangements in scanned images containing aggressive B-cell lymphoma with morphology and phenotype consistent with diffuse large B-cell lymphoma (DLBCL) or high-grade B-cell lymphoma (HGBL). In such instances, an HPS negative label may indicate that MYC gene rearrangement is ruled out, and an HPS positive label may indicate that MYC gene rearrangement is not ruled out.

This allows the biomarker detection system 100 to identify cases that can safely be omitted from laborious and expensive molecular cytogenetic testing and enable pathologists to focus on the molecular characterization of remaining cases using more advanced and comprehensive molecular testing, as needed (e.g., FISH testing using multiple probes against IG heavy and light chain loci, cancer genomic profiling using next-generation sequencing). Given the cost and limitations of current FISH testing, a combined, sequential testing approach that begins with a digital screening test (via the biomarker detection system 100) with high sensitivity to rule out MYC-R, and is followed by a molecular confirmation test with high specificity (e.g., a FISH test) may decrease resource consumption and increase overall testing performance.

In some examples, the biomarker detection system 100 may be used to predict MYC gene rearrangement in Burkitt lymphoma to avoid FISH testing or to predict gene expression signatures such as the double-hit signature (DHITsig) or molecular high-grade (MGH) signature in DLBCL/HGBL to avoid expression profiling. In further examples, the biomarker detection system 100 utilizes HPS as a biomarker that characterizes/identifies a specific subpopulation of DLBCL/HGBL patients with a shared biology/pathophysiology and further enables applications, such as patient selection or stratification in clinical trials.

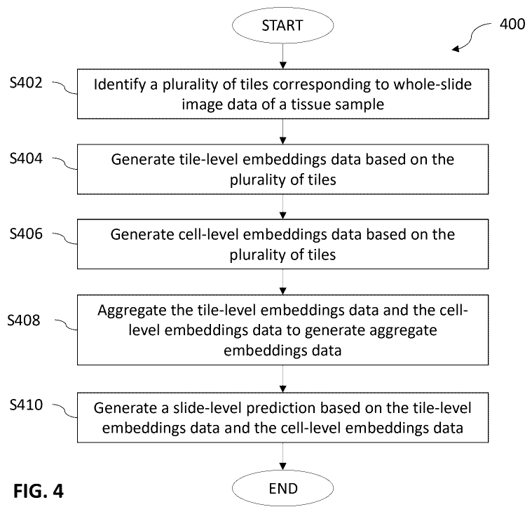

FIG. 4 is a flow diagram illustrating a process 400 of detecting a biomarker by the biomarker detection system 100, according to some embodiments of the present disclosure.

In some embodiments, the biomarker detection system 100 identifies a plurality of tiles 115 corresponding to whole-slide image data 10 of a tissue sample (S402) by receiving the whole-slide image data 10 corresponding to the tissue sample, and extracting the plurality of tiles 115 from the whole-slide image data 10. The whole-slide image data 10 may include at least one a digitized WSI 11 of the tissue sample of a patient that is stained with hematoxylin and eosin (H&E) dyes or a region-of-interest (ROI) map 12.

In some embodiments, the biomarker detection system 100 generates tile-level embeddings data 121 based on the plurality of tiles (e.g., normalized tiles) 115 (S404). In doing so, the second model of the tile-level analyzer 120 may generate a plurality of tile-level feature vectors 121 based on the plurality of tiles 115. The second model may include at least one of a residual network (ResNet) or a transformer network.

The biomarker detection system 100 also generates (e.g., concurrently generates) cell-level embeddings data 137 based on the plurality of tiles (S406). In doing so, the detection system 100 may extract a plurality of cell patches 133 based on the plurality of tiles 115, and may generate the cell-level embeddings data 137 based on the plurality of cell patches 133.

In some examples, extraction of the cell-level embeddings data 137 includes detecting, by the segmentation model 140, a plurality of cells in each one of the plurality of tiles 115, and generating the plurality of cell patches 133 based on the plurality of tiles 115 and the plurality of cells in each one of the plurality of tiles. Each cell patch 133 may include a portion of one of the plurality of tiles 115 containing a single cell.

In some examples, generation of the cell-level embeddings data 137 includes generating, by the third model of the cell feature generator 134, a plurality of cell-level feature vectors 135 based on the plurality of cell patches 133, and combining the plurality of cell-level feature vectors 135 to generate the cell-level embeddings data 137. The cell-level embeddings data 137 may include a plurality of embedding vectors.

In some embodiments, the biomarker detection system 100 aggregates the tile-level embeddings data 121 and the cell-level embeddings data 137 to generate aggregate embeddings data 151 (S408). In doing so, the detection system concatenating the tile-level embeddings data and the cell-level embeddings data to generate the aggregate embeddings data.

According to some embodiments, the biomarker detection system 100 (e.g., the biomarker predictor 160) generates a slide-level prediction 20 based on the aggregate embeddings data (i.e., based on the tile-level embeddings data 121 and the cell-level embeddings data 137) (S410). The slide-level prediction 20 may indicate presence or absence of the biomarker (e.g., MYC-R) in the tissue sample.

In some examples, the output of the biomarker detection system 100 may be utilized to rule out MYC rearrangements in scanned images containing aggressive B-cell lymphoma with morphology and phenotype consistent with diffuse large B-cell lymphoma (DLBCL) or high-grade B-cell lymphoma (HGBL). In some examples, the biomarker detection system 100 may be used to predict gene expression signatures such as the double-hit signature (DHITsig) or molecular high-grade (MGH) signature in DLBCL/HGBL to avoid expression profiling. In further examples, the biomarker detection system 100 utilizes HPS as a biomarker that characterizes/identifies a specific subpopulation of DLBCL/HGBL patients with a shared biology/pathophysiology. The predictive ability of the detection system 100 may also enable applications such as patient selection or stratification in clinical trials.

According to various embodiments of the present disclosure, the biomarker detection system 100 is implemented using one or more processing circuits or electronic circuits configured to perform various operations as described above. Types of electronic circuits may include a central processing unit (CPU), a graphics processing unit (GPU), an artificial intelligence (AI) accelerator (e.g., a vector processor, which may include vector arithmetic logic units configured efficiently perform operations common to neural networks, such dot products and softmax), a field programmable gate array (FPGA), an application specific integrated circuit (ASIC), a digital signal processor (DSP), or the like. For example, in some circumstances, aspects of embodiments of the present disclosure are implemented in program instructions that are stored in a non-volatile computer readable memory where, when executed by the electronic circuit (e.g., a CPU, a GPU, an AI accelerator, or combinations thereof), perform the operations described. The operations performed by the biomarker detection system 100 may be performed by a single electronic circuit (e.g., a single CPU, a single GPU, or the like) or may be allocated between multiple electronic circuits (e.g., multiple GPUs or a CPU in conjunction with a GPU). The multiple electronic circuits may be local to one another (e.g., located on a same die, located within a same package, or located within a same embedded device or computer system) and/or may be remote from one other (e.g., in communication over a network such as a local personal area network such as Bluetooth®, over a local area network such as a local wired and/or wireless network, and/or over wide area network such as the internet, such a case where some operations are performed locally and other operations are performed on a server hosted by a cloud computing service). One or more electronic circuits operating to implement the biomarker detection system 100 may be referred to herein as a computer or a computer system, which may include memory storing instructions that, when executed by the one or more electronic circuits, implement the systems and methods described herein.

It will be understood that, although the terms “first”, “second”, “third”, etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section discussed below could be termed a second element, component, region, layer or section, without departing from the spirit and scope of the inventive concept.

The terminology used herein is for the purpose of describing particular embodiments and is not intended to be limiting of the inventive concept. As used herein, the singular forms “a” and “an” are intended to include the plural forms as well, unless the context clearly indicates otherwise. It will be further understood that the terms “include,” “including,” “comprises,” “comprising,” “has,” “have,” and “having,” when used in this specification, specify the presence of stated features, integers, steps, operations, elements, and/or components, but do not preclude the presence or addition of one or more other features, integers, steps, operations, elements, components, and/or groups thereof.

As used herein, the term “and/or” includes any and all combinations of one or more of the associated listed items. For example, the expression “A and/or B” denotes A, B, or A and B. Expressions such as “one or more of” and “at least one of,” when preceding a list of elements, modify the entire list of elements and do not modify the individual elements of the list. For example, the expression “one or more of A, B, and C,” “at least one of A, B, or C,” “at least one of A, B, and C,” and “at least one selected from the group consisting of A, B, and C” indicates only A, only B, only C, both A and B, both A and C, both B and C, or all of A, B, and C.

Further, the use of “may” when describing embodiments of the inventive concept refers to “one or more embodiments of the inventive concept.” Also, the term “exemplary” is intended to refer to an example or illustration.

It will be understood that when an element or layer is referred to as being “on”, “connected to”, “coupled to”, or “adjacent” another element or layer, it can be directly on, connected to, coupled to, or adjacent the other element or layer, or one or more intervening elements or layers may be present. When an element or layer is referred to as being “directly on,” “directly connected to”, “directly coupled to”, “in contact with”, “in direct contact with”, or “immediately adjacent” another element or layer, there are no intervening elements or layers present.

As used herein, the term “substantially,” “about,” and similar terms are used as terms of approximation and not as terms of degree, and are intended to account for the inherent variations in measured or calculated values that would be recognized by those of ordinary skill in the art.

As used herein, the terms “use,” “using,” and “used” may be considered synonymous with the terms “utilize,” “utilizing,” and “utilized,” respectively.

When one or more embodiments may be implemented differently, a specific process order may be performed differently from the described order. For example, (i) the disclosed operations of a process are merely examples, and may involve various additional operations not explicitly covered, and (ii) the temporal order of the operations may be varied.

Unless otherwise defined, all terms (including technical and scientific terms) used herein have the same meaning as commonly understood by one of ordinary skill in the art to which the present inventive concept belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Although aspects of some example embodiments of the system and method for biomarker detection have been described and illustrated herein, various modifications and variations may be implemented, as would be understood by a person having ordinary skill in the art, without departing from the spirit and scope of embodiments according to the present disclosure. Accordingly, it is to be understood that a pathology slide manufacturing system and method according to the principles of the present disclosure may be embodiment other than as specifically described herein. The disclosure is also defined in the following claims, and equivalents thereof.

What is claimed is:

1. A method of detecting a biomarker by a detection system based on machine learning, the method comprising:
identifying, by the detection system, a plurality of tiles corresponding to whole-slide image data of a tissue sample;
generating, by the detection system, tile-level embeddings data based on the plurality of tiles;
generating, by the detection system, cell-level embeddings data based on the plurality of tiles; and
generating, by the detection system, a slide-level prediction based on the tile-level embeddings data and the cell-level embeddings data, the slide-level prediction indicating presence or absence of the biomarker in the tissue sample.

2. The method of claim 1, wherein the identifying the plurality of tiles comprises:
receiving, by the detection system, the whole-slide image data corresponding to the tissue sample; and
extracting, by the detection system, the plurality of tiles from the whole-slide image data.

3. The method of claim 1, wherein the whole-slide image data comprises at least one a digitized image of the tissue sample of a patient that is stained with hematoxylin and eosin (H&E) dyes or a region-of-interest (ROI) map.

4. The method of claim 1, further comprising:
performing stain normalizing, by the detection system, based on the plurality of tiles to generate a plurality of normalized tiles,
wherein the generating the tile-level embeddings data comprises:
generating, by the detection system, the tile-level embeddings data from the plurality of normalized tiles.

5. The method of claim 4, wherein the performing stain normalizing comprises:
generating, by a first model of the detection system, the plurality of normalized tiles based on the plurality of tiles.

6. The method of claim 5, wherein the first model comprises a fully convolutional neural network.

7. The method of claim 1, wherein the generating the tile-level embeddings data comprises:
generating, by a second model of the detection system, a plurality of tile-level feature vectors based on the plurality of tiles, and
wherein a number of the tile-level feature vectors corresponds to a number of the tiles.

8. The method of claim 7, wherein the second model comprises at least one of a residual network (ResNet) or a transformer network, and
wherein the number of the tile-level feature vectors is a same as the number of the tiles.

9. The method of claim 1, wherein the generating the cell-level embeddings data comprises:
extracting, by the detection system, a plurality of cell patches based on the plurality of tiles; and
generating, by the detection system, the cell-level embeddings data based on the plurality of cell patches.

10. The method of claim 9, wherein the extracting the plurality of cell patches comprises:
detecting, by a segmentation model of the detection system, a plurality of cells in each one of the plurality of tiles; and
generating, by the detection system, the plurality of cell patches based on the plurality of tiles and the plurality of cells in each one of the plurality of tiles, a cell patch of the plurality of cell patches comprises a portion of one of the plurality of tiles containing a single cell of the plurality of cells.

11. The method of claim 10, wherein the generating the plurality of cell patches comprises:
generating, by the detection system, the plurality of cell patches from a plurality of normalized tiles corresponding to the plurality of tiles.

12. The method of claim 10, wherein the generating the cell-level embeddings data comprises:
generating, by a third model of the detection system, a plurality of cell-level feature vectors based on the plurality of cell patches, and
wherein a number of the cell-level feature vectors corresponds to a number of the plurality of tiles and a number of the cell patches.

13. The method of claim 12, wherein the third model comprises at least one of a residual network (ResNet) or a transformer network, and
wherein the number of the cell-level feature vectors is a number of the plurality of tiles multiplied by a number of the cell patches.

14. The method of claim 12, wherein the generating the cell-level embeddings data further comprises:
combining, by the detection system, the plurality of cell-level feature vectors to generate the cell-level embeddings data, the cell-level embeddings data comprising a plurality of embedding vectors, and
wherein a number of the embedding vectors corresponds to a number of the plurality of tiles.

15. The method of claim 14, wherein an embedding vector of the plurality of embedding vectors comprises an average of a number of the plurality of cell-level feature vectors and a standard deviation of the number of the plurality of cell-level feature vectors.

16. The method of claim 1, further comprising:
aggregating, by the detection system, the tile-level embeddings data and the cell-level embeddings data to generate aggregate embeddings data,
wherein generating the slide-level prediction is by a fourth model of the detection system and is based on the aggregate embeddings data.

17. The method of claim 16, wherein the aggregating the tile-level embeddings data and the cell-level embeddings data comprises:
concatenating, by the detection system, the tile-level embeddings data and the cell-level embeddings data to generate the aggregate embeddings data, a vector length of the aggregate embeddings data is equal to a sum of vector lengths of the tile-level embeddings data and the cell-level embeddings data.

18. The method of claim 16, wherein the fourth model comprises at least one of a multiple-instance learning (MIL) network, an attention-based MIL (AMIL) network, or a transformer.

19. The method of claim 1, wherein the slide-level prediction comprises an MYC-driven high-grade B-cell lymphoma (HGBL) signature.

20. The method of claim 1, further comprising:
transmitting the slide-level prediction to a display device for display to a user.

21. A detection system for detecting a biomarker, the detection system comprising:
a processor; and
a memory storing instructions that, when executed on the processor, cause the processor to perform:
identifying a plurality of tiles corresponding to whole-slide image data of a tissue sample;
generating tile-level embeddings data based on the plurality of tiles;
generating cell-level embeddings data based on the plurality of tiles; and
generating a slide-level prediction based on the tile-level embeddings data and the cell-level embeddings data, the slide-level prediction indicating presence or absence of the biomarker in the tissue sample.

22. The detection system of claim 21, wherein the identifying the plurality of tiles comprises:
receiving the whole-slide image data corresponding to the tissue sample; and
extracting the plurality of tiles from the whole-slide image data, and
wherein the whole-slide image data comprises at least one a digitized image of the tissue sample of a patient that is stained with hematoxylin and eosin (H&E) dyes or a region-of-interest (ROI) map.

23. The detection system of claim 21, wherein the generating the tile-level embeddings data comprises:
generating a plurality of tile-level feature vectors based on the plurality of tiles, and
wherein a number of the tile-level feature vectors corresponds to a number of the tiles.

24. The detection system of claim 21, further comprising:
performing stain normalizing based on the plurality of tiles to generate a plurality of normalized tiles,
wherein the generating the tile-level embeddings data comprises:
generating the tile-level embeddings data from the plurality of normalized tiles.

25. The detection system of claim 21, wherein the generating the cell-level embeddings data comprises:
extracting a plurality of cell patches based on the plurality of tiles; and
generating the cell-level embeddings data based on the plurality of cell patches.

26. The detection system of claim 25, wherein the extracting the plurality of cell patches comprises:
detecting a plurality of cells in each one of the plurality of tiles; and
generating the plurality of cell patches based on the plurality of tiles and the plurality of cells in each one of the plurality of tiles, a cell patch of the plurality of cell patches comprises a portion of one of the plurality of tiles containing a single cell of the plurality of cells.

27. The detection system of claim 26, wherein the generating the cell-level embeddings data comprises:
generating a plurality of cell-level feature vectors based on the plurality of cell patches, and
wherein a number of the cell-level feature vectors corresponds to a number of the plurality of tiles and a number of the cell patches.

28. The detection system of claim 27, wherein the generating the cell-level embeddings data further comprises:
combining the plurality of cell-level feature vectors to generate the cell-level embeddings data, the cell-level embeddings data comprising a plurality of embedding vectors,
wherein a number of the embedding vectors corresponds to a number of the plurality of tiles, and
wherein an embedding vector of the plurality of embedding vectors comprises an average of a number of the plurality of cell-level feature vectors and a standard deviation of the number of the plurality of cell-level feature vectors.

29. The detection system of claim 21, further comprising:
aggregating the tile-level embeddings data and the cell-level embeddings data to generate aggregate embeddings data,
wherein generating the slide-level prediction is by a fourth model of the detection system and is based on the aggregate embeddings data.

30. The detection system of claim 29, wherein the aggregating the tile-level embeddings data and the cell-level embeddings data comprises:
concatenating the tile-level embeddings data and the cell-level embeddings data to generate the aggregate embeddings data, a vector length of the aggregate embeddings data is equal to a sum of vector lengths of the tile-level embeddings data and the cell-level embeddings data.
